(19) United States
(12) Patent Application Publication
(10) Pub. No.: US 2026/0290508 A1
(43) Pub. Date: Sep. 24, 2026

(54) METHOD FOR DETERMINING INTERDETECTOR DELAY BETWEEN ANALYTICAL INSTRUMENT AND FRACTION COLLECTOR

(71) Applicant: Wyatt Technology. LLC, Goleta, CA (US)

(72) Inventor: Steven P. Trainoff, Santa Barbara, CA (US)

(21) Appl. No.: 19/576,487

(22) Filed: Mar. 24, 2026

Related U.S. Application Data
(60) Provisional application No. 63/776,749, filed on Mar. 24, 2025.

(51) Int. Cl. G16B 40/10

(52) U.S. Cl. CPC G16B 40/10

(57) ABSTRACT

Described is a method for determining an interdetector delay comprising performing an analysis of a sample in a flow of an analytical instrument, the analytical instrument being in communication with a fraction manager and having a detector configured to generate a first signal proportional to a concentration of the sample in the flow; dispensing, using the fraction manager, the flow from the analytical instrument into a plurality of vials, wherein a content of at least some of the vials includes a portion of the sample; for each of the vials, performing an analysis of the content of the vial using the analytical instrument to generate a second signal proportional to the concentration of the sample in the flow of the analytical instrument; for each of the second signals, integrating the second signal over time to determine an integrated value; determining fit parameters for the integrated values; and determining an interdetector delay time based on the first signal and the fit parameters.

### RELATED APPLICATION

This application is a non-provisional patent application claiming priority to U.S. Provisional Patent Application No. 63/776,749, filed Mar. 24, 2025, titled “Method for Determining Interdetector Delay Between Analytical Instrument and Fraction Collector,” which is incorporated herein by reference in its entirety.

### FIELD OF THE INVENTION

The disclosed technology relates generally to analytical systems employing a fraction collector or fraction manager. More particularly, the technology relates to fraction collectors or fraction managers used in combination with an analytical instrument such as a size exclusion chromatography (SEC) system or a field flow fractionation (FFF) system.

### BACKGROUND

Fraction collectors or Fraction Managers are often used after SEC or FFF separations to collect aliquots of subfractions into vials or well plates. There are two popular methods to determine when to start collecting fractions and when to change vials. The first method is time-based in which the start time of collections is set before the fractionation run is started. Then fractions are collected on a pre-determined schedule. This method works well when the fractionation process is highly repeatable and has been previously characterized. The second method is more flexible and uses an analytical instrument to act as a trigger to start collecting the fraction. The trigger can be as simple as setting a threshold on one analytical detector in the analysis chain. A popular choice is to use an ultraviolet (UV) or refractive index (RI) detector to generate the trigger. Collection begins when the absorption signal or differential refractive index signal crosses a pre-determined threshold value. This method is often referred to as peak-based collection. The criterion can be more sophisticated by using multiple detector signals to derive quantities of fundamental interest such as molar mass, or size which are computed in real time and used as a trigger signal.

A benefit of using a peak-based trigger method is that drifts in the fractionation process can be accommodated. If fractions from many replicate runs are to be collected, it is likely that the arrival time of the trigger will be subtly different from run to run. For example, the peak can arrive at 1.0 min. in the first run, 1.01 min. in the second run, and 1.02 min. in the third run. The time-based collection method causes the fractions collected in each vial to be slightly different. The trigger-based collection method automatically adapts to the shifting peak arrival time.

One problem with the peak-based collection method is that the sample takes a finite time to travel from the triggering detector to the fraction collector needle. In general, when multiple detectors are used to derive a trigger signal, there is a delay associated with the time that the derived signal triggers and when the sample arrives at the fraction collector needle. Additionally, there can be a time delay between the beginning of an injection, and when the fraction manager starts the vial collection. Some analytical instrument and fraction collector systems include a dedicated sensor inside the fraction collector that is used to determine the “delay time” between the analytical system detector and the fraction collector needle. In other analytical instrument and fraction collector combinations, there may be no additional detector in the fraction collector. In such systems, the analytical instrument detector is the only detector available for determining the time delay.

### SUMMARY

In one aspect, a method for determining an interdetector delay time comprises (a) performing an analysis of a sample in a flow of an analytical instrument, the analytical instrument being in communication with a fraction manager and having a detector configured to generate a first signal proportional to a concentration of the sample in the flow; (b) dispensing, using the fraction manager, the flow from the analytical instrument into a plurality of vials, wherein a content of at least some of the vials includes a portion of the sample; (c) for each of the vials, performing an analysis of the contents of the vial using the analytical instrument to generate a second signal proportional to the concentration of the sample in the flow of the analytical instrument; (d) for each of the second signals, integrating the second signal over time to determine an integrated value; (e) determining fit parameters for the integrated values; and (f) determining an interdetector delay time based on the first signal and the fit parameters.

In another aspect, a computer-implemented method for determining an interdetector delay time comprises: (a) performing, by a computer processor, an analysis of a sample in a flow of an analytical instrument, the analytical instrument being in communication with a fraction manager and having a detector configured to generate a first signal proportional to a concentration of the sample in the flow; (b) dispensing, using the fraction manager, the flow from the analytical instrument into a plurality of vials, wherein a content of at least some of the vials includes a portion of the sample; (c) for each of the vials, performing, by a computer processor, an analysis of the contents of the vial using the analytical instrument to generate a second signal proportional to the concentration of the sample in the flow of the analytical instrument; (d) for each of the second signals, integrating, by a computer processor, the second signal over time to determine an integrated value; (e) determining, by a computer processor, fit parameters for the integrated values; and (f) determining, by a computer processor, an interdetector delay time based on the first signal and the fit parameters.

In another aspect, a non-transitory computer-readable medium storing instructions that, when executed by one or more processors, cause the processors to perform a method for determining an interdetector delay time, the method comprising: (a) controlling an analytical instrument to perform an analysis of a sample in a flow of the analytical instrument, the analytical instrument being in communication with a fraction manager and having a detector configured to generate a first signal proportional to a concentration of the sample in the flow; (b) controlling the fraction manager to dispense the flow from the analytical instrument into a plurality of vials, wherein a content of at least some of the vials includes a portion of the sample; (c) for each of the vials, directing the analytical instrument to perform an analysis of the contents of the vial to generate a second signal proportional to the concentration of the sample in the flow of the analytical instrument; (d) integrating, for each of the second signals, the second signal over time to determine an integrated value; (e) determining fit parameters for the integrated values; and (f) determining an interdetector delay time based on the first signal and the fit parameters.

### BRIEF DESCRIPTION OF THE DRAWINGS

The above and further advantages of this invention may be better understood by referring to the following description in conjunction with the accompanying drawings, in which like numerals indicate like structural elements and features in the various figures. For clarity, not every element may be labeled in every figure. The drawings are not necessarily to scale, emphasis instead being placed upon illustrating the principles of the invention.

FIG. 1 shows an overlay of a UV signal as measured by an inline analytical UV detector and a signal generated by an internal sensor inside the fraction collector.

FIG. 2 shows the two signals of FIG. 1 shifted with respect to each other to correct for the transit time between the analytical UV detector and the internal sensor of the fraction collector.

FIG. 3 is a block diagram of a system in which embodiments of the present inventive concept can be practiced.

FIG. 4 is a flowchart representation of an embodiment of a method to measure an interdetector delay time.

FIG. 5 is a signal generated by a UV detector in response to a calibration sample.

FIG. 6 shows a number of signals from the UV detector with each signal corresponding to an injection of a corresponding one of a series of collected fractions of the calibration sample of FIG. 5.

FIG. 7 shows a plot of the computed measured mass values as determined from the area under each peak shown in FIG. 6.

FIG. 8 graphically depicts a least square fit of the values shown in FIG. 7 overlaid with values derived by integrating the original signal in FIG. 5.

FIG. 9 shows an example of baseline shift wander that is evident by comparing the measurements of injections that do not include any sample with the first vial that contains sample.

### DETAILED DESCRIPTION

Reference in the specification to an embodiment or example means that a particular feature, structure or characteristic described in connection with the embodiment or example is included in at least one embodiment or example of the teaching. References to a particular embodiment or example within the specification do not necessarily all refer to the same embodiment or example.

The present teaching will now be described in detail with reference to exemplary embodiments or examples thereof as shown in the accompanying drawings. While the present teaching is described in conjunction with various embodiments and examples, it is not intended that the present teaching be limited to such embodiments and examples. On the contrary, the present teaching encompasses various alternatives, modifications, and equivalents, as will be appreciated by those of skill in the art. Moreover, features illustrated or described for one embodiment or example may be combined with features for one or more other embodiments or examples. Those of ordinary skill having access to the teaching herein will recognize additional implementations, modifications, and embodiments, as well as other fields of use, which are within the scope of the present disclosure as described herein.

In brief overview, embodiments and examples disclosed herein are directed to a method for determining an interdetector delay between an analytical instrument and a fraction manager configured to receive the flow from the analytical instrument. A detector in the analytical instrument generates a signal that is linearly proportional to the concentration of the sample in the flow from the instrument to the fraction manager. Two or more fractions are collected into corresponding fraction vials in the fraction manager and subsequently these vials are transferred to an autosampler and serially injected back into the analytical instrument. The mass of the sample (or a value proportional to the mass of the sample) from each collection vial is computed by integrating over the peak in the detector signal as generated for the corresponding injection. A least squares fit determines the optimal interdetector delay time based on the computed data. The determined value for the interdetector delay time can be used to delay the start of fraction collection to ensure that a triggered sample is collected in the first vial. Advantageously, if the sample is fractionated according to the method so that the portions of the sample in the vials are nearly monodisperse, a variety of detectors are suitable for use in the analytical instrument. By way of non-limiting examples, the detector may be a UV detector, a RI detector or a Multi-Angle Static Light Scattering (MALS) detector and used within its linear detection range.

Various terminology is used in the description below. As used herein, both fraction manager and fraction collector are used to describe systems used to collect fractions from a separation system. Similarly, reference is made to a triggering detector as well as an analytical instrument detector and it should be understood that both terms are used interchangeably and that such detectors are used to generate a signal proportional to a concentration of a sample passing through the detector.

As discussed above, a problem with a peak-based method for fraction collection is that the sample takes a finite time to travel from a triggering detector in the analytical instrument to the fraction collector needle. A dedicated detector inside the fraction collector can be used to determine the delay time between the analytical instrument detector and the fraction collector needle; however, if the fraction collector has no dedicated internal detector, the delay time between the triggering detector and the time the sample arrives at the fraction collector needle requires determination by some other means.

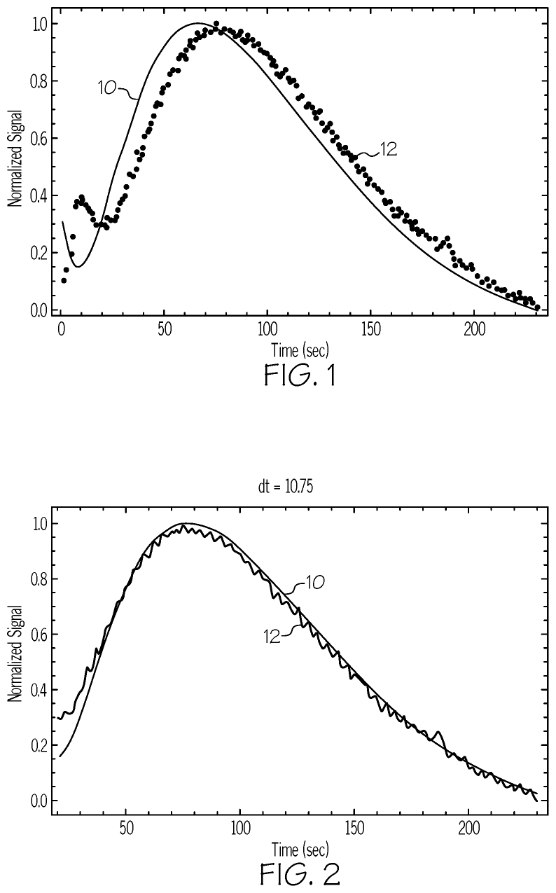

The interdetector delay time delay Δt in a system having a triggering detector in the analytical instrument and a separate internal detector in the fraction collector is related to the interdetector volume IV defined between the triggering detector and the fraction collector needle as IV=Δt f, where f is the volumetric flow rate. For example, FIG. 1 shows an overlay of a UV signal 10 as measured by an inline analytical UV detector and the signal 12 provided by an internal UV sensor inside the fraction collector. The delay between the two signals 10 and 12 is due to the transit time through the flow path between the UV detector and the fraction collector. Simply shifting the two signals relative to each other by 10.75 seconds, as shown in FIG. 2, corrects for the transit time. In particular, the time delay to align the two signals was 10.75 sec. Thus, if the inline UV detector, delayed by 10.75 sec, is used as the trigger, the sample will arrive at the fraction collector needle when the collection into a vial begins.

The same method cannot be used for a fraction collector that does not include an internal sensor. Conventionally, the user estimates the delay time between the triggering signal and the fraction manager by calculation. This is accomplished by summing the internal volumes of all the tubing and intervening instruments, and then converting into a delay time by using Δt=IV/f, where IV is the computed interdetector (delay) volume. Since there can be several intervening instruments between the trigging detector and the fraction collector, this method is susceptible to substantial calculation error. Additionally, if there is a time delay between the start time between the injection and the analytical instrument, this additional delay time is not accounted for by the simple volume calculation.

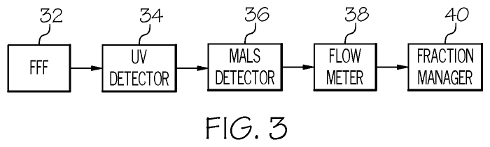

A common configuration shown in the block diagram in FIG. 3 uses a FFF system 32 followed by UV detector 34, followed by MALS detector 36, to a fraction collector 40, although many other configurations are possible. For example, the system may be a Neon™ Eclipse™ FFF-MALS system available from Waters/Wyatt Technologies of Santa Barbara, CA. The system uses an inline flow meter 38 to regulate the detector flow after the analytical instruments so that the sample returns to the system for this flow rate measurement before passing the sample to the fraction manager 40. If the UV detector 34 is used as the trigger, the delay associated with the volume of the tubing from the UV detector 34 to the MALS detector 36 (e.g., DAWN™ light scattering detector from Waters/Wyatt Technologies of Santa Barbara, CA), the internal volume of the MALS detector 36, the volume inside the flow meter 38 and the volume in the tubing from the flow meter 38 to the fraction manager 40 is calculated. The resulting calculation can only be considered a rough estimate of the true interdetector delay volume. Alternatively, if the MALS detector 36 is used as the trigger, the volume from the MALS detector 36 to the fraction manager 40 must be determined.

Described below is a simple automated method to measure the interdetector delay time in situ, regardless of which detector is used as the trigger detector or how many instruments or lengths of tubing are between the trigger detector and the fraction collector.

The method to determine the interdetector delay time is based on injecting a calibration sample into an analytical instrument and collecting the sample exiting the instrument into two or more fraction vials. During the instrument analysis, the signal from the triggering detector (or a composite signal like molar mass or particle size) is recorded as a function of time. In the following description of an embodiment of the method, the configuration includes a Neon Eclipse FFF-MALS system referenced above as the fractionator and the triggering detector is a UV detector. More generally, other embodiments of the method can utilize other analytical instruments and fraction collectors.

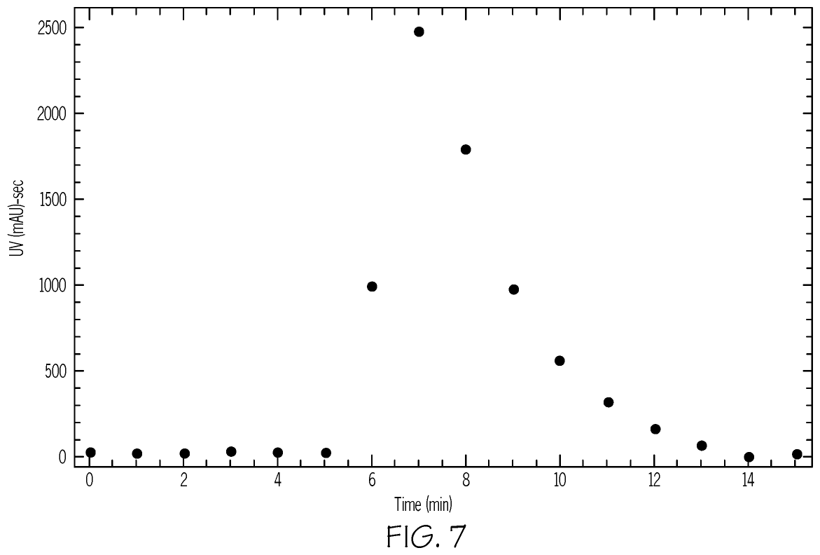

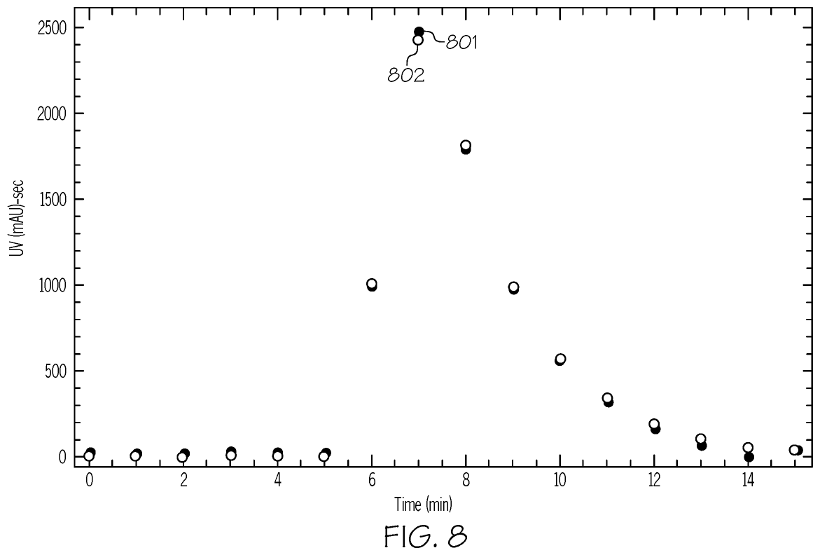

The described embodiment uses a FFF collection of a calibration sample to determine the interdetector delay time Δt. If the analytical instrument includes a UV detector, then a UV active sample such as bovine serum albumin (BSA) can be used. For the calibration, there is no need to fractionate the sample, and there is no requirement that the concentration or molar mass of the calibration sample is known. The only requirement is that the signal from the detector include a peak having a large amplitude with enough volume that portions of it can be captured into separate vials. The fraction collector is then configured to collect the peak into a number of vials using a time-based collection. Preferably, each vial is collected for a constant collection time, but this is not required so long at the start and stop time of each vial collection is known. The initial collected vials may precede the arrival of the sample at the fraction collector and the final collected vials may not contain any sample as their collections may occur after the full sample has been processed by the fraction collector. This ensures that the full sample peak is collected into a subset of the total number of vials. Notwithstanding this, there is no strict requirement that the full sample peak be collected, but an analysis may be performed where there are sample-free vials before and after the calibration peak. For example, as shown in FIG. 7, there are only one or two points in the tail. The analysis would still apply if these points were disregarded.

During this initial FFF collection of the calibration sample, the detector signal UV(t) is used to determine the concentration as a function of time c(t). The concentration c(t) is integrated over the time interval associated with each vial i to get

$${m}_{i} ( Δ t ) = {\int }_{{t}_{i}}^{{t}_{i + 1}} c ( t - Δ t ) dt ,$$

where mᵢ(Δt) is the predicted mass that will be deposited into vial i if the delay time between the trigger device and the fraction collector is Δt, tᵢ is the start time of fraction i, and Δt is the (unknown) delay time. The time tᵢ can be determined from the collection volume V such that tᵢ=iV/f where f is the detector flow rate. This assumes that there are no gaps between subsequent fractions. If there is some sample lost between vial i and the subsequent vial i+1, then the upper integration limit time limit tᵢ₊₁ should be replaced with the time of the end of fraction i.

Alternatively, the determination of the concentration c(t) is not a necessary step as the integration can be performed instead using the trigger signal UV(t) after zero correction.

The fraction manager collects the sample into the vials. Ideally the fraction volumes are chosen to be small compared to the peak width, so that that the peak shape is resolved. If there is insufficient sample in each vial for reinjection (see below), multiple runs may be performed using multiple triggers to accumulate more sample into the vials.

The vials are then transferred to the autosampler to await re-injection. A series of runs of the FFF system are performed with each run corresponding to an injection volume v from each of the vials. If the vial includes a portion of the calibration samples, the run will result in a signal having a peak. Each peak is integrated over time to determine the total mass, as seen by the trigger detector, for the injection. The resulting data yields a measurement of the mass of the calibration sample that was detected into each of the vials. The data of the measured mass in each vial m′ᵢ and the predicted mass in each vial mᵢ (Δt) can be fit to determine the optimal value of Δt. Because only a portion of each vial is reinjected, the values m′ᵢ will smaller than the total mass that was deposited into each vial. This is accommodated in the fit by including a floating scale constant α and baseline offset b. χ² is computed as

$${χ}^{2} ( a , b , Δ t ) = {\sum }_{i = 1}^{N} {( a {m}_{i} ( Δ t ) + b - {m}_{i}^{′} )}^{2}$$

Where the best fit parameters α, b and Δt are determined by minimizing χ². The interdetector delay time returned from the fitter is Δt. Although α is not a required parameter, its value should be nearly

$$\frac{v}{V} ,$$

which is the portion of the vial volume that was reinjected. This ratio can be used as a check on the fit. The parameter b is included to account for differences in detector baselines between the initial run and the later vial reinjection runs.

The injection performed for each vial is made only to determine the total mass within the injection. Like the initial calibration sample injection, there is no need to fractionate the subsequent injections, although if such fractionations occur there is no effect on the results. For the Neon Eclipse configuration referenced above, the injections can be sped up by using a method that has zero cross flow (i.e., just void volume) and no dilution control module flow (i.e., the detector flow equals the channel flow). Consequently, the dilution from running the reinjected samples from the vials through the system is minimized, thereby improving the accuracy of the method.

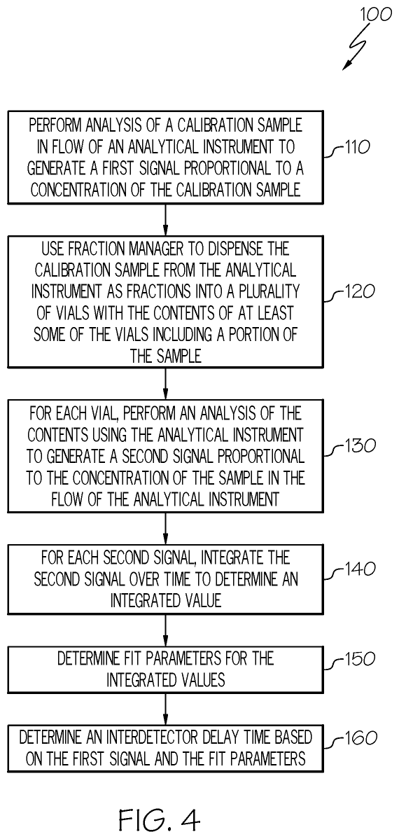

Referring to FIG. 4, a flowchart representation of a generalized embodiment of a method 100 for determining an interdetector delay time is shown. The method 100 can be performed using various types of analytical instruments and fraction collectors as are known to those of skill in the art. The method 100 includes performing (step 110) an analysis of a calibration sample in a flow of an analytical instrument in communication with a fraction manager. The analytical instrument has a detector configured to generate a first signal proportional to a concentration of the sample in the instrument flow. The fraction manager dispenses (step 120) the flow from the analytical instrument into a plurality of vials. At least some of the contents of the vials include a portion (fraction) of the sample present in the flow from the instrument. For each vial, an analysis of the contents of the vial is performed (step 130) by the analytical instrument to generate a second signal proportional to the concentration of the sample in the flow from the analytical instrument. Each of the second signals is integrated (step 140) over time to determine an integrated value and these integrated values are used to determine (step 150) fit parameters, as described in more detail in the example below. The interdetector delay time is then determined (step 160) from the first signal and the fit parameters.

### Example

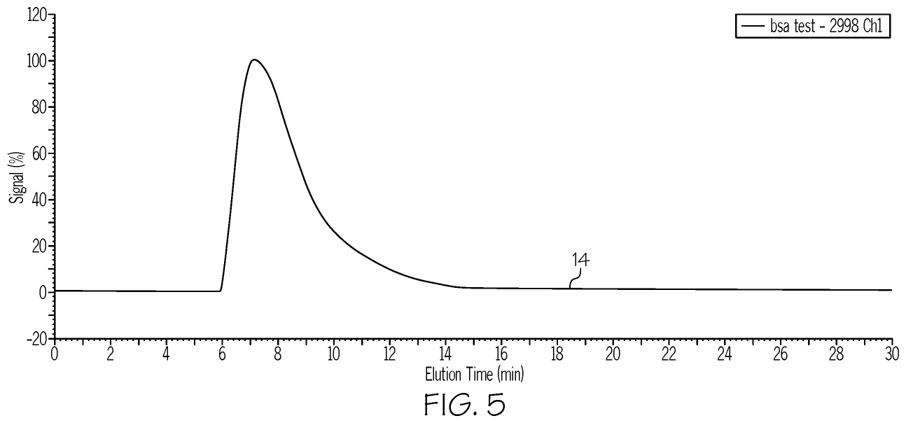

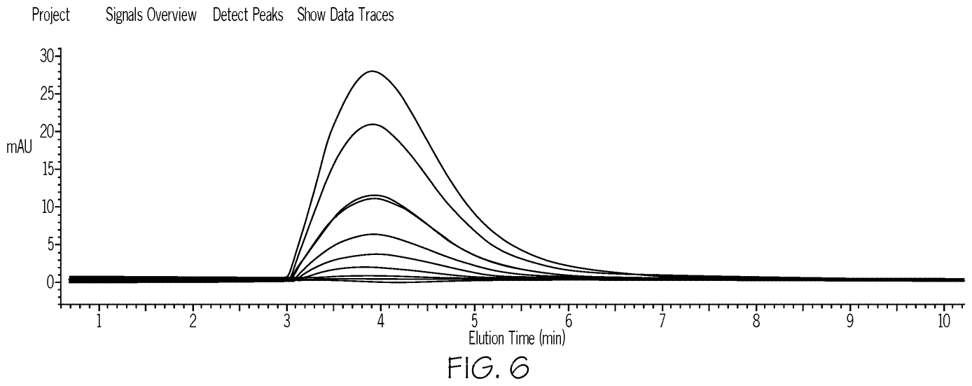

The method described above was applied to a Neon Eclipse system plumbed directly to a UV detector and a fraction manager. The calibration sample used to determine the interdetector delay time was 1.0 mg/ml Sigma BSA prepared in 50 mM phosphate buffered saline (PBS). The channel was bypassed with a 5-port union and BSA was injected directly into the UV detector, resulting in trace 14 shown in FIG. 5. The sample was collected into a series of vials using a time-based collection starting at t=0 and switching vials every 500 μl (1.0 min). A total of 16 vials were used for the delay determination. After the collection, the vials were transferred into the autosampler. Subsequently, 50 μl from each vial was injected to the system. The corresponding peaks from each vial injection are shown overlaid in FIG. 6. The area under each peak was computed to determine the measured mass values m′ᵢ, which are shown in FIG. 7, where the peak areas have units of mAU-sec.

Subsequently, a least square fit was performed to find the optimal values of α, b, and Δt, as described above. The resulting fit is shown in FIG. 8 where the two plots are the original m′ᵢ points of FIG. 1 (labeled 801) and the rescaled trigger data a mᵢ (Δt)+b 802, which has the same units (mAU-sec). For this data set the optimal values are α=25.569, b=−15.907 and Δt=0.1751 min (10.506 sec). Thus, for a subsequent experiment of a fractionated sample a trigger can be set to initiate when the UV detector signal passes a specified threshold. The sample that causes the threshold crossing will arrive in the fraction collector needle 10.506 sec later. Thus, starting fraction collection upon expiration of this delay results in the sample that caused the trigger to be collected into the first vial. This process can be repeated for subsequent peaks causing threshold crossings with the corresponding samples being collected into other vials by implementing the same delay value.

### Enhanced Method

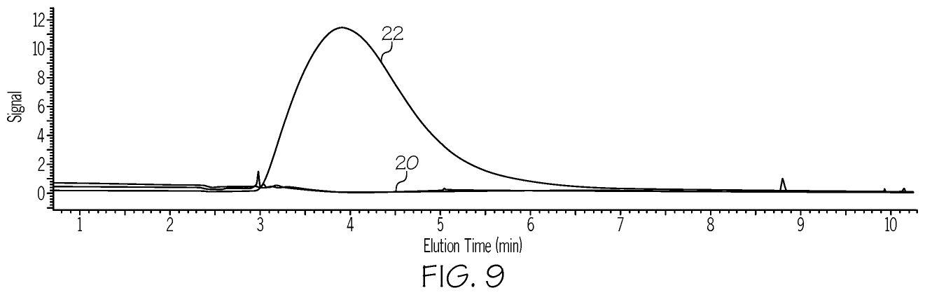

In the example described above, the vials collected before 4 minutes (see FIG. 8) in the example above are completely empty of sample and consist of pure solvent. However, in practice, there is a small baseline shift wander that can be associated with pressure changes in the system. This baseline drift can be seen by comparing the measurements of some of the “empty” injections 20 with the first vial that contains sample 22, as shown in FIG. 9.

When computing the integral of the trace to determine the sample mass in each vial, the results are more accurate if the trace 20 of one or more of the empty vials is used as a blank run. Therefore, the measured mass m′ᵢ can be computed as

$${m}_{{i}^{´}}^{′} = {\int }_{peak} ( {c}_{i} ( t ) - {c}_{blank} ( t ) ) dt$$

where cᵢ(t) is the concentration determined from vial i and c_{blank} (t) is the signal from the run used as the blank injection. The integration is performed over the peak region after setting a suitable baseline and corrects for any non-ideal effects from the baseline wander.

While various examples have been shown and described, the description is intended to be exemplary, rather than limiting and it should be understood by those of ordinary skill in the art that various changes in form and detail may be made therein without departing from the scope of the invention as recited in the accompanying claims.

What is claimed is:

1. A method for determining an interdetector delay time, the method comprising:
(a) performing an analysis of a sample in a flow of an analytical instrument, the analytical instrument being in communication with a fraction manager and having a detector configured to generate a first signal proportional to a concentration of the sample in the flow;
(b) dispensing, using the fraction manager, the flow from the analytical instrument into a plurality of vials, wherein a content of the vials includes a portion of the sample;
(c) for each of the vials, performing an analysis of the content of the vials using the analytical instrument to generate a second signal proportional to the concentration of the sample in the flow of the analytical instrument;
(d) for each of the second signals, integrating the second signal over time to determine an integrated value;
(e) determining fit parameters for the integrated values; and
(f) determining an interdetector delay time based on the first signal and the fit parameters.

2. The method of claim 1, wherein the fit parameters include at least one of a floating fit constant and a baseline offset.

3. The method of claim 1, wherein steps (a) and (b) are repeated to increase a volume of the content of the vials.

4. The method of claim 1, wherein the performing an analysis according to step (c) comprises injecting into the analytical instrument only a partial volume of the content of the vials.

5. The method of claim 1, wherein the analytical instrument is one of a field flow fractionator, a size exclusion separation device and a liquid chromatography system.

6. The method of claim 1, wherein one or more vials of the plurality of vials does not include any sample, the method further comprising determining a baseline wander signal based on at least one of the second signals associated with the one or more vials that do not include the any sample.

7. The method of claim 6, further comprising subtracting the baseline wander signal from each of the second signals to generate a corrected second signal and wherein step (d) comprises integrating the corrected second signals over time to determine the integrated values.

8. A computer-implemented method for determining an interdetector delay time, the method comprising:
(a) performing, by a computer processor, an analysis of a sample in a flow of an analytical instrument, the analytical instrument being in communication with a fraction manager and having a detector configured to generate a first signal proportional to a concentration of the sample in the flow;
(b) dispensing, using the fraction manager, the flow from the analytical instrument into a plurality of vials, wherein a content of at least some of the vials includes a portion of the sample;
(c) for each of the vials, performing, by a computer processor, an analysis of the content of the vials using the analytical instrument to generate a second signal proportional to the concentration of the sample in the flow of the analytical instrument;
(d) for each of the second signals, integrating, by a computer processor, the second signal over time to determine an integrated value;
(e) determining, by a computer processor, fit parameters for the integrated values; and
(f) determining, by a computer processor, an interdetector delay time based on the first signal and the fit parameters.

9. The computer-implemented method of claim 8, wherein the fit parameters include at least one of a floating fit constant and a baseline offset.

10. The computer-implemented method of claim 8, wherein steps (a) and (b) are repeated to increase a volume of the content of the vials.

11. The computer-implemented method of claim 8, wherein the performing an analysis according to step (c) comprises injecting into the analytical instrument only a partial volume of the content of the vials.

12. The computer-implemented method of claim 8, wherein the analytical instrument is one of a field flow fractionator, a size exclusion separation device and a liquid chromatography system.

13. The computer-implemented method of claim 8, wherein one or more vials of the plurality of vials does not include any sample, the method further comprising determining a baseline wander signal based on at least one of the second signals associated with the one or more vials that do not include the any sample.

14. The computer-implemented method of claim 13, further comprising subtracting the baseline wander signal from each of the second signals to generate a corrected second signal and wherein step (d) comprises integrating the corrected second signals over time to determine the integrated values.

15. A non-transitory computer-readable medium storing instructions that, when executed by one or more processors, cause the processors to perform a method for determining an interdetector delay time, the method comprising:
(a) controlling an analytical instrument to perform an analysis of a sample in a flow of the analytical instrument, the analytical instrument being in communication with a fraction manager and having a detector configured to generate a first signal proportional to a concentration of the sample in the flow;
(b) controlling the fraction manager to dispense the flow from the analytical instrument into a plurality of vials, wherein a content of the vials includes a portion of the sample;
(c) for each of the vials, directing the analytical instrument to perform an analysis of the content of the vials to generate a second signal proportional to the concentration of the sample in the flow of the analytical instrument;
(d) integrating, for each of the second signals, the second signal over time to determine an integrated value;
(e) determining fit parameters for the integrated values; and
(f) determining an interdetector delay time based on the first signal and the fit parameters.

16. The non-transitory computer-readable medium of claim 15, wherein the fit parameters include at least one of a floating fit constant and a baseline offset.

17. The non-transitory computer-readable medium of claim 15, wherein steps (a) and (b) further comprise repeating the analysis and dispensing operations to increase a volume of the content of the vials.

18. The non-transitory computer-readable medium of claim 15, wherein the analytical instrument is one of a field-flow fractionator, a size-exclusion separation device, or a liquid chromatography system.

19. The non-transitory computer-readable medium of claim 1, wherein one or more vials of the plurality of vials does not include any sample, the medium further storing instructions that, when executed, cause the processor to determine a baseline-wander signal based on at least one of the second signals associated with the one or more vials that do not include the any sample.

20. The non-transitory computer-readable medium of claim 19, further storing instructions that, when executed, cause the processor to: subtract the baseline-wander signal from each of the second signals to generate a corrected second signal, and wherein integrating according to step (d) comprises integrating the corrected second signals over time to determine the integrated values.
